(19) United States
(12) Patent Application Publication
(10) Pub. No.: US 2020/0210983 A1
(43) Pub. Date: Jul. 2, 2020

(54) Generating Online Share Slips with Clickable Web Links for Electronic Share Transfers for Preferentially Matched Members with E-Service Capabilities

(71) Applicant: Samaritan Ministries International, Inc., Peoria, IL (US)

(72) Inventor: Wayne Black, Normal, IL (US)

(73) Assignee: Samaritan Ministries International, Inc., Peoria, IL (US)

(21) Appl. No.: 16/577,110

(22) Filed: Sep. 20, 2019

Related U.S. Application Data
(60) Provisional application No. 62/734,508, filed on Sep. 21, 2018.

(51) Int. Cl. G06Q 20/22; G06Q 40/02; G06Q 20/10; G16H 40/00

(52) U.S. Cl. CPC G06Q 20/223; G16H 40/00; G06Q 20/102; G06Q 40/02

(57) ABSTRACT

A system and method for generating an online share slip with a clickable web link for a plurality of members of a peer-to-peer needs sharing community. A share preference group and a share amount are assigned to each of the plurality of members corresponding to the member's preferred method of sending an outbound share. The needs are assigned a needs preference group. The allocation component prioritizes the matching of shares with needs having a preference group. Preference groups can be an online e-payment service to effectuate the transfer of the funds to maximize the speed and efficiency of transfer shares for members in need.

### CROSS REFERENCES

Not Applicable.

### REFERENCE TO CDS

Not Applicable.

### FIELD OF THE INVENTION

The present disclosure relates to generating online share slips with clickable web links for electronic share transfers for preferentially matched members with e-service capabilities, enabling an increased number of members in a peer-to-peer health care needs sharing organization to engage in electronic share transfers.

### BACKGROUND

Healthcare sharing is an alternative to healthcare insurance products. Healthcare sharing is a type of a mutual benefit society where members commit to sharing in a portion of the medical needs of the other members. An organization can take an active role in receiving and disbursing the funds or a more passive role by providing share information to the members to engage in a peer-to-peer direct sharing model.

In a health care sharing model, generally, a member pays a periodic share, commonly monthly. When a member has a qualifying healthcare expense, the member may submit that expense as a need. The healthcare sharing organization allocates one or more other members to meet that need. The need can be met by the other members directly, in a peer-to-peer direct sharing model, or indirectly where the members send their share money to the healthcare sharing organization to collectively pool and distribute to the member in need.

### SUMMARY

E-Sharing provides members the opportunity to send and receive shares through a payment platform they may already use in addition to sharing through postal mail. Sharing electronically is possible when both the sending and receiving member use the same payment platform.

I discovered a process to preferentially connect members who had a compatible share transfer method during the periodic allocation of shares. Some members are more technologically savvy, using computers, cell phones, tables, and other network connected electronic devices to communicate and manage money. Other members are less technologically savvy or merely have a preference for paper mail and checks. By preferentially displaying a graphical online share slip with a clickable web link to members set up to receive the shares via a digital, network-based solution to other members with similar capabilities, the overall membership has a better, more efficient sharing experience.

I also recognized that the time for a member to receive the shares may be decreased when members who are able to pay their shares electronically are matched with member desiring to receive shares electronically. Paper mail is slower than electronic mail, and sending checks through the mail is slower than sending funds using an online transfer service. In order for a member with a need to receive a faster online transfer, the member with the need is setup to receive the online transfer and the member sending the share is setup to send the online transfer. If the receiving member is not setup to receive the online transfer, then a member willing to send a share through online transfer is unable to take advantage of the faster online transfer. Similarly, if a member with a need is setup to receive an online transfer but the share sending member is not setup for online transfers, then the share must be transferred by postal mail.

Members may add and edit their payment links through a member portal having a graphical user interface. The member selects their preferred e-service payment method and enters their individualized payment links for one or more e-services. When the member has a need, the sharing members receive a share slip through email or the member portal. The share slip has a clickable web link to send the Share electronically instead of through postal mail.

I discovered that preferentially matching groups having the largest amount of total need dollars ensures that a greater number of members receive a share slip with a clickable web link to initiate an electronic payment of their share. The needs are sorted by total group dollar. The sharing preference group having the highest total dollar amount is selected. The largest needs within the need group having the highest total dollar amount is selected for allocation. The largest share, or the most closely matching share size, may be matched to the selected need. In this way, a larger number of sharing members receive an online share slip to send their share through an e-service.

I also recognized that maximizing the number of members sharing through electronic share transfer services minimizes the overall time between a member's need submission and receipt of all of their shares. The time to send a paper check through the mail is inherently time consuming: manually transposing the share amount from the share slip to the check, manually transposing the name and address of member to the check and envelope, affixing proper postage to the envelope, depositing the share in a mailbox, waiting for the postal service to deliver the share to the receiving member, and waiting for the member to retrieve the share from member's mailbox. The receiving member must then open the mail, manually complete a shares received checklist, and deposit the check with the member's bank. This can be especially burdensome when members have large needs, necessitating tens or hundreds of shares. Electronic share transfer systems can be almost instantaneous. As soon as the member clicks the appropriate web link the receiving user may receive the share.

Another advantage of the current disclosure is the generation of a web link that sends the share amount parameter to the online transfer service. When members send shares direct in the peer-to-peer model, the members commonly handwrite a personal check or manually enter the share dollar amount into an online bill payment or other check-printing interface. I discovered that displaying a web link in an online share slip allows the exact share dollar value to be automatically transferred to the online transfer service. By sending the share amount parameter through a clickable web link, the frequency of user error may be decreased compared with the member transposing numbers from a paper share slip on to a check.

Another advantage of the current disclosure is that the sending member does not have to manually transpose the receiving member's name and address correctly. When members send shares direct in the peer-to-peer model, the members commonly handwrite the receiving member's name and address on an envelope. I discovered that displaying a web link to a member's electronic sharing system profile enables the receiving member to control the receipt address through the electronic sharing system's profile. The receiving member can configure their electronic sharing system profile to deliver notices to their preferred email address and deposit electronically transferred funds to their preferred bank account. The receiving member can manage all of their received shares remotely. This enables a hospitalized member to manage the member's shares received from a hospital bed. Similarly, in situations where there is a natural disaster, the member may have medical needs arising from the natural disaster. The member may have to evacuate or be set up in a transient living situation, which can be difficult for the member to send checks or receive paper mail at a physical address. Maximizing the sending of shares through online share slips and electronic share transfers enables members to continue to participate in a healthcare sharing organization despite a transient living situation.

While the present disclosure is focused upon healthcare sharing, the same principles may apply to the sharing of other household expenses, income protection such as from disability, legal liability protection, and property protection.

A principal object of the present invention is to provide an improved peer-to-peer needs sharing system and method which facilitates user-friendly and efficient generation of computerized needs sharing for the near instantaneous electronic transfer of shares utilizing equipment utilizing devices in the possession of the member, such as smart phones, tablets, or personal computers.

It is understood that other embodiments will become readily apparent to those skilled in the art from the following detailed description, wherein various embodiments are shown and described by way of illustration only. As will be realized, the concepts are capable of other and different embodiments and their several details are capable of modification in various other respects, all without departing from the spirit and scope of what is claimed as the invention. Accordingly, the drawings and detailed description are to be regarded as illustrative in nature and not as restrictive.

### BRIEF DESCRIPTION OF DRAWINGS

Aspects are illustrated by way of example, and not by way of limitation, in the accompanying drawings, wherein:

FIG. 1 depicts a schematic illustration of peer-to-peer need sharing.

FIG. 2 depicts various matches between members who are or are not setup for electronic share transfers.

FIG. 3 depicts a schematic illustration of storing a member's share preference service priority.

FIG. 4 depicts a flow chart for the allocation engine preferentially matching members setup for electronic share transfers with receiving members setup to receive electronic share transfers.

FIG. 5 depicts a graphical user interface for an online share slip with clickable web links enabling the sending member to send a share through an electronic transfer service.

FIG. 6 depicts an additional information display and confirmation for the online share slip of FIG. 5.

FIG. 7 depicts an additional information display and confirmation for the online share slip of FIG. 5.

FIG. 8 depicts a schematic illustration of ranking multiple e-service providers according to a member preference rank.

FIG. 9 depicts a graphical user interface enabling a member to select a sharing preference between multiple e-services.

FIG. 10 depicts a flow chart for an allocation engine preferentially matching e-shares with e-needs and then matching regular shares with regular needs.

FIG. 11 depicts a flow chart for an allocation engine to allocate shares within a shares analysis queue to selected needs from a needs analysis queue.

FIG. 12 depicts a flow chart for an allocation engine to perform a matching set.

FIG. 13 depicts a flow chart for an allocation engine to preferentially match shares from a share preference group identical to the need before matching other compatible share preference groups.

FIG. 14 depicts a flow chart for the allocation engine preferentially matching members setup for electronic share transfers with receiving members setup to receive electronic share transfers.

FIG. 15 depicts a flow chart for an allocation engine to determine the compatibility of the online share slip category as a compatible share preference group based on a selected need preference group.

FIG. 16 depicts a sample data set of qualifying member needs for a period.

FIG. 17 depicts a sample data set of sharing category associated with memberships.

FIG. 18 depicts a sample data set of need category associated with memberships.

FIG. 19 depicts a sample data set of an allocation having matched various shares from memberships to a need.

### DETAILED DESCRIPTION

Allocation is the process of matching membership shares to member needs. The goal is to assign each share with a need. The allocation process is run periodically upon all of the needs submitted for the previous period. The period is commonly a monthly period, but could also be annually, quarterly, weekly, daily, or hourly depending on the number of members, regularity of needs, size of needs, and the members' need for prompt reimbursement. The number and amount of shares available to meet the needs may be affected by the number of members, the dollar amount of each member's share, deferment of a portion of the shares to the organization for administrative cost, and member credits. The allocation engine is a computer-based algorithm used to balance the shares and needs and to match each share with a need.

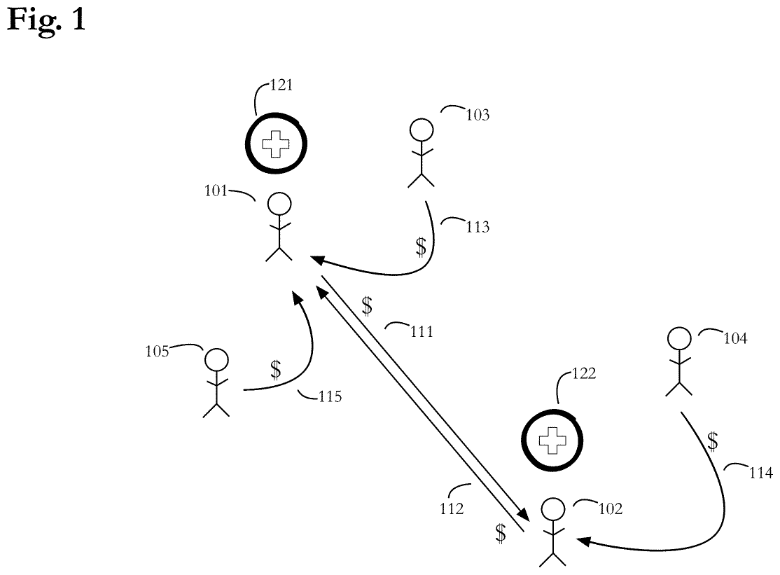

A hypothetical healthcare sharing organization is shown schematically in FIG. 1. The healthcare sharing organization comprises member 101, member 102, member 103, member 104, and member 105. Each member sends a share each period. Member 101 had medical need 121 and member 102 had medical need 122. Member 101 and Member 102 submit their need to the healthcare sharing organization. The health care sharing organization determines if the need qualifies for sharing under the organization's guidelines. The shares are allocated as follows: Member 101 sends share 111 to Member 102; Member 102 sends share 112 to Member 101; Member 103 sends share 113 to Member 101; Member 104 sends share 114 to Member 102; and Member 105 sends share 115 to Member 101. Each member sends a share to another member who had a need. This allows Member 101 and Member 102 to pay their medical expenses with the funds they receive from their fellow members. Each member bears a portion of the medical needs 121, 122.

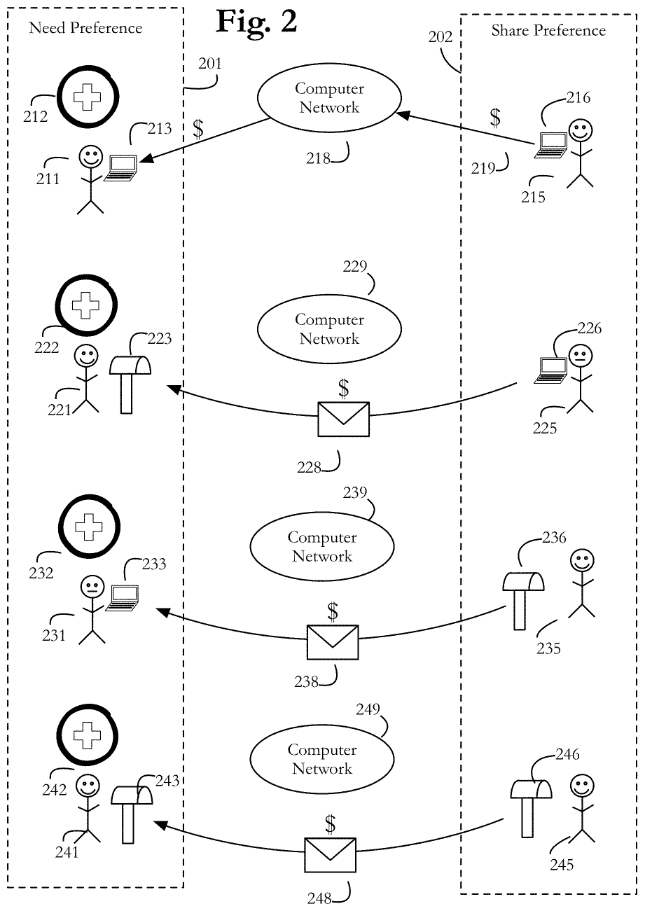

As shown in FIG. 2, a member can send or receive a share to another member in a variety of ways. Members having a need are shown in box 201. Members assigned to send a share are shown in box 202. Member 211 has medical need 212. Member 211 is setup to receive shares via an online transfer service, as indicated by icon 213. Online transfer services include Peer-to-peer payment applications and web portals like PayPal®, Square® Cash App™, PayPal® Venmo™, Google® Pay™, Apple® Pay Cash™. Banks may offer online transfer services using web portals or apps such as Zelle®. Member 215 is also set up for online transfer service, as indicated by icon 216. Member 215 share is allocated by the allocation engine to Member 211's medical need 212. Member 215 receives an online share slip enabling member to send the share electronically. Member 215 sends the share 219 through online transfer service via a computer network connection 218 between member 211 and member 215. Member 211 receives the share 219 through the online transfer service via the computer network connection 218.

When a member with a need is not setup to receive online share transfers, then a member sharing who is setup for online share transfers may be inconvenienced. Member 221 has medical need 222. Member 221 is not setup to receive shares via an online transfer service. Rather, Member 221 can only receive shares by paper mail, as indicated by icon 223. Member 225 is set up for online transfer service, as indicated by icon 226. Member's 225 share is allocated by the allocation engine to Member's 221 medical need 222. Member 225 may receive an online share slip, but it will not have a clickable web link to an e-service. Member 225 sends the share 228 by writing a check, manually transposing the share amount from the share slip to the check, transposing the name and address of member 221 to the check and envelope, affixing proper postage to the envelope, and mailing the check. After several days, Member 221 receives the share 228 through the paper mail service. Even though member 225 is setup for online transfer service through computer network connection 229, member 221 cannot receive an online share transfer. Therefore, the default transfer method of paper mail is required of member 225.

Similarly, when a member with a need is setup to receive online share transfers, but a member allocated to send the share is not setup for online share transfers, then the receiving member may be inconvenienced. Member 231 has medical need 232. Member 231 is setup to receive shares via an online transfer service, as indicated by icon 233. Member 235 is not set up for online transfer service, as indicated by icon 236. Member's 235 share is allocated by the allocation engine to Member's 231 medical need 222. Member 235 sends the share 238 by writing and mailing a check. Member 231 receives the share 238 through the paper mail service. Member 231 would have received share 238 sooner if the share sending member 235 was setup to send through an online share transfer service. Even though member 231 is setup to receive online transfer service through computer network connection 239, member 235 cannot send an online share transfer. Therefore, the default transfer method of paper mail is required of member 235.

When a member with a need is not setup to receive online share transfers and a member allocated to send the share who is also not setup for online share transfers, then neither party is inconvenienced. Member 241 has medical need 242. Member 241 is not setup to receive shares via an online transfer service, as indicated by icon 243. Member 245 is also not set up for online transfer service, as indicated by icon 246. Member's 245 share is allocated by the allocation engine to Member's 241 medical need 242. Member 245 receives a paper share slip. Member 245 sends the share 248 by writing and mailing a check. Member 241 receives the share 248 through the paper mail service and computer network connection 249 may not be utilized.

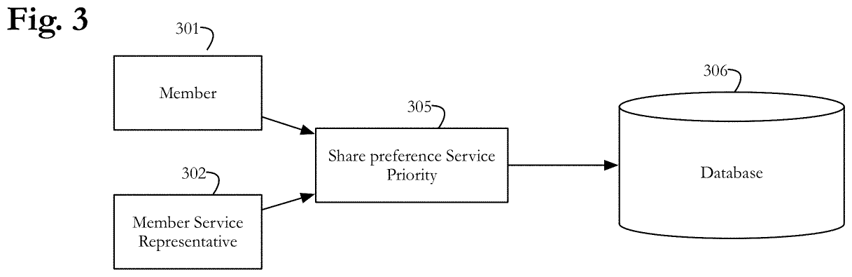

Members may select one or more preferred online transfer services to send shares and to receive shares. As shown in FIG. 3, the member 301 or the member service representative 302 may select a share preference service priority 305. The category order for share preference service priority 305 may be a selected electronic transfer service (“e-service”), then online share slip, then paper checks. The category order may then be stored in a database 306. In one embodiment, the selected e-service is a single service provider. In another embodiment, the selected e-service has multiple service provider options.

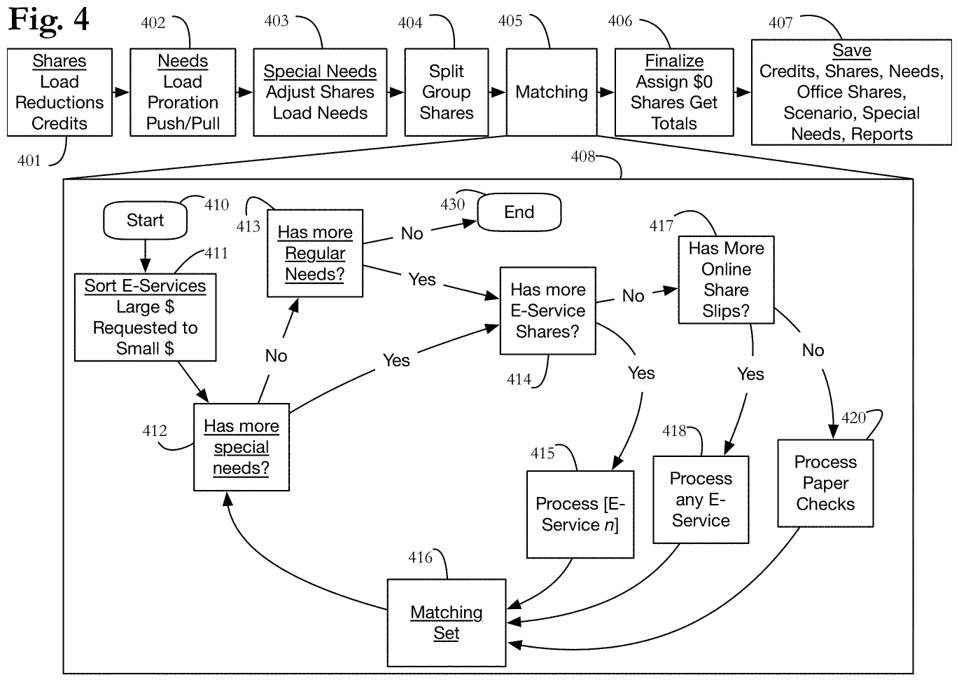

In order to increase the efficiency of sharing between members with compatible need sharing transfer service preferences, the allocation engine preferentially matches members who have expressed a preference to similar electronic transfer services together. In order to increase the percentage of members successfully match, the allocation engine prioritizes the matching of the electronic transfer service having the largest dollar amount of total needs to shares matching the electronic transfer service, as shown in FIG. 4.

In one embodiment, the first step is to load shares, as shown in step 401. All shares are loaded into a shares analysis queue in the analysis engine. Any reductions are applied to the shares once they have been loaded. Reductions occur when the total dollar amount of available shares exceeds the total dollar amount of needs. The result is that the shares—with the exception of shares sent to the sharing organization for administrative overhead—are reduced by a predetermined percentage. Credits are also applied to the shares at this step. Credits can be applied to shares to reward certain member behavior, such as referring other members to the sharing organization. Both reductions and credits reduce the dollar amount that a member sends in their share. A credit reduces the dollar amount of a specific member's share, whereas the reduction is applied to most or all shares.

The next step is the needs step, as shown in step 402. All needs for the current period are loaded into a needs analysis queue. At this time the total amount that is shareable is calculated. Proration is a reduction of each need by a predetermined percentage, which may happen when the total dollar amount of needs exceeds the total dollar amount of shares. Proration affects the total amount that is shareable.

The allocation engine calculates and displays the total dollar amount of shares available for the current period, the total dollar amount of loaded needs for the current period, and the total dollar amount of needs set to load in the subsequent period. The analyst can pull certain needs, or a portion thereof, from the subsequent period into the current period. Alternatively, the analyst can push certain needs, or a portion thereof, from the current period into a subsequent period. This provides the analyst with the discretionary ability to affect the total dollar amount of loaded needs for a current period. By pulling one or more additional needs into the current period, when that need would otherwise not be loaded until the subsequent period, the total dollar amount of the needs for the current period increases. This may be advantageous to fully utilize the total dollar amount of the shares for the current period. Another advantage to pulling needs into the current period is to reduce the number of shares needed for the subsequent period, thereby preventing a situation where the total dollar amount of the needs in a subsequent period exceeds the total dollar amount of the shares available in that period. A need added to the push list reduces the total amount shareable for the current period. An advantage to pushing a need to a subsequent period allows the needs in the current period to be more fully shared when the total dollar amount of needs in the current period exceed the total dollar amount of shares available in that period. The analyst can push an entire share, a specific dollar amount, or a predetermined percentage. If the total dollar amount of a need is less than a defined minimum shareable amount, then that need is removed from the need queue.

In the embodiment shown in FIG. 4, the healthcare sharing organization can utilize a separate sharing structure for special needs, as shown in step 403. This separate sharing structure allows members to voluntarily elect to participate in the sending and receiving of shares related to special needs. In one example, regular needs are limited to $250,000.00 and a special need is a need whose total dollar amount exceeds $250,000.00, specifically the amount of the need that exceeds $250,000.00. If the member elects to participate in the special needs program, then that member shares in the portion of a need that exceeds $250,000.00 with other members who have elected to participate in the special needs program. The member participating in the example special program only sends this additional share if there are qualifying needs. If the special program shares are not needed, the participating member holds the funds in a reserve to build up a special program participation balance. This balance can grow over the years if there the total dollar amount of the qualifying special needs is less than the cumulative set aside for the participating members over a given period. In this example, the member commits to set aside a particular amount of money per year, as determined by the program guidelines to contribute an additional share portion to the periodic share for special needs. Any special needs that have been approved for sharing are loaded into a special needs queue. In one embodiment, the allocation engine calculates the amount of special needs relative to the number of members participating in the special needs program to form a special needs participation share amount. The special needs participation share amount is added to the share of each participating member as an adjusted share amount.

Each membership or group of memberships can choose to send more than one share each period, resulting in a split share, as shown in step 404. The member service representative may associate a split share parameter with a membership willing to send multiple shares. The allocation engine generates a number of smaller shares based on the split share parameter corresponding with the number of shares the member is willing to send. The split calculator creates copies of the spilt share and adjusts the share amount accordingly, so the two shares may be of unequal amounts but will add up to the same dollar amount as a single share for that member. For large share groups there is a configurable maximum share dollar amount that will automatically force a share to be split, such that the share amount of each split share does not exceed the maximum share dollar.

The shares in the shares analysis queue are then allocated to the needs in the needs analysis queue in matching step 405. The matching step 405 is expanded in box 408, discussed below. As shown in step 406, after the matching step has completed, the share allocation is finalized. Any remaining shares that have a zero dollar amount are assigned. And a total sum of shares assigned is calculated. According to step 407, the routine is saved which creates a record of credits, shares, needs, office shares, scenario information, special needs, and reports.

Box 408 shows an expanded detail flow chart for the matching step 405. On the start 410, the allocation engine begins the matching step 405. The allocation engine sorts all needs marked as e-service, pursuant to step 411. Needs are sorted from largest dollar amount to smallest dollar amount. Except, however, a collector need may be added to the bottom of the queue otherwise out of the queue's order. Alternatively, the needs may be sorted by chronology, severity of a need, manually by the analyst, or according to a calculated metric that measures a member's involvement within the ministry, such as length of membership or timeliness of sending shares.

The allocation engine may prioritize the processing of special needs, as shown in step 412. The allocation engine processes needs in the special needs group with shares in the special needs group. In this example, only members who are in special needs program share to a qualifying special need. Therefore the special needs are processed in separate cycles. In another example, the allocation engine does not discriminate between a special needs member and a non-special needs member. In this first round, special needs program shares are matched to special needs in the same way that regular shares are matched to regular needs, which is discussed below.

Once all the special e-service needs are matched, the allocation proceeds to match regular e-service needs with regular shares, according to step 413. The allocation engine selects the largest regular e-service need from the needs analysis queue. The system may look for the smallest regular e-service share that is greater than the need amount that has not yet been met. If no share from the share analysis queue will meet the remaining need amount completely, the largest share available in the share analysis queue is selected.

Once there is a preliminary match between a share and a need, the allocation engine performs matching quality controls. The system prevents a share that belongs to the need's membership from being assigned to the need. This prevents a membership from sharing with itself. For split shares, the system is configured to assign only one share per membership or group to a selected need. This prevents a membership from being asked to send two shares to the same member.

After a preliminary match and passing the error controls, the selected share is then assigned to the selected need and the amount needed is reduced by the share dollar amount. Then the share is removed from the shares analysis queue.

If there are additional e-service shares, as shown in Step 414, the system continues to apply e-service shares from the shares analysis queue to the selected need from the same e-service group, according to step 415. The selected share is then assigned to the selected need and the amount needed is reduced by the share dollar amount, as shown in step 416. Then the share is removed from the shares analysis queue.

The system continues to match e-service shares, steps 413-416, to the selected need until the total amount of shares is greater than or equal to the need amount. If the total amount of the shares is greater than the need amount, the difference between the need amount and the sum of the shares is considered overage. The system searches the shares analysis queue to ensure no shares are available that are smaller than the overage. If there is such a share available, the allocation engine removes the last added share putting the last added share back into the shares analysis queue to be assigned to a different need.

Once the shares analysis queue is exhausted of a selected e-service group shares, then the system proceeds to the next e-service group having the next largest dollar amount of needs. The allocation engine iteratively processes each e-service group from the largest dollar amount to the smallest dollar amount, until either the selected e-service group's needs or shares is exhausted.

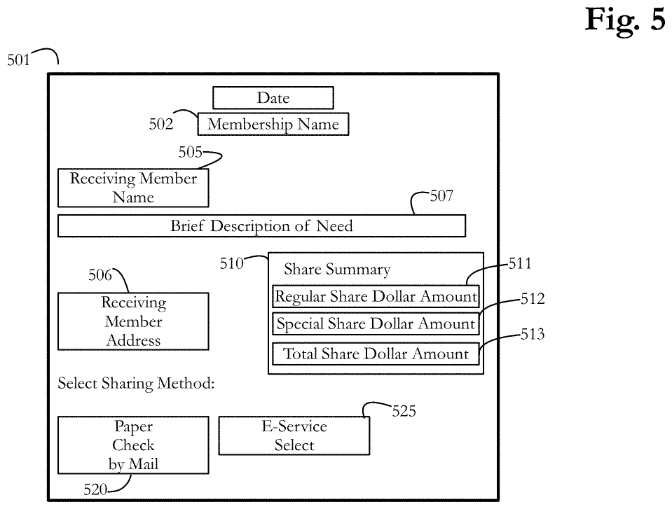

Once the e-service group needs have been matched with the e-service group shares, then the allocation engine matches any non-regular share in the shares analysis queue to memberships that receive an online share slip, according to step 417. The online share slip 501 is an electronic graphical user display associated with a specific membership, as shown in FIG. 5 and discussed below. The allocation engine proceeds to match any remaining e-service needs with shares from the online share slip group, according to step 418. Members who receive online share slips are presented with clickable web links for e-services, and therefore may participate in the receiving member's preferred e-service even if the sending member has not indicated their participation in that e-service.

Once the queue has been exhausted of either all of the e-service needs from the needs analysis queue or the online share slip shares from the shares analysis queue, then the system proceeds to process paper checks, according to step 420.

The allocation engine continues to process needs from the needs analysis queue and shares from the shares analysis queue until all shares are assigned or all needs are met, according to step 430. The analyst may adjust various settings, such as pulls and pushes, until all the shares have been assigned and only the collector need is not met. Once the analyst is satisfied with the allocation, the analyst selects to commit the allocation to production. The allocation data is pushed to the production system to generate share slips, both printed share slips and online share slips.

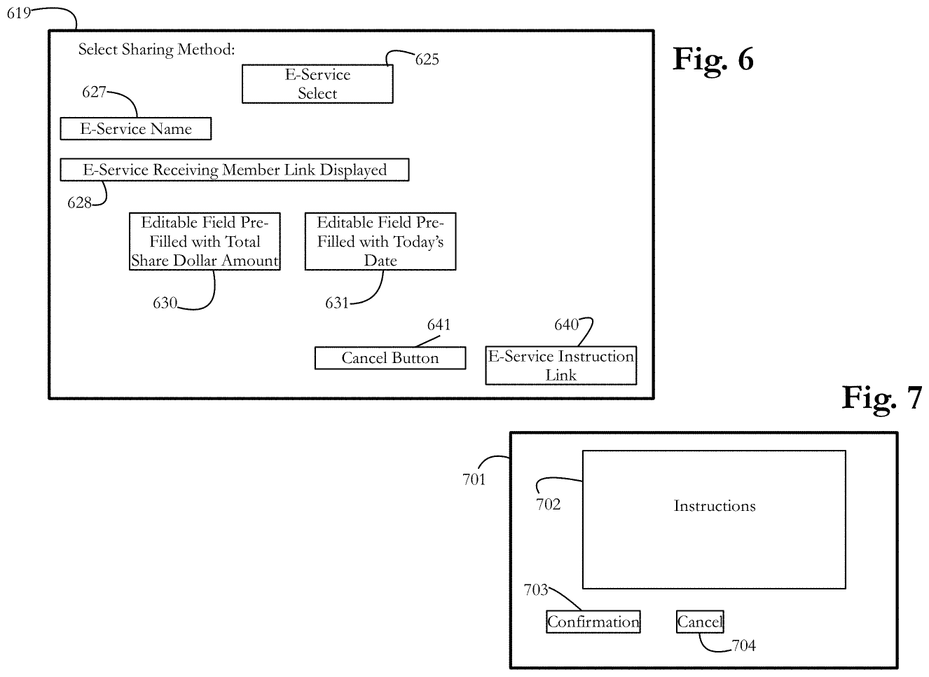

The online share slip 501 gives share information for the member's assigned share, as shown in FIG. 5. The online share slip 501 may indicate the membership name 502, receiving member's name 505, receiving member's mailing address 506, and a brief description of the need 507. The online share slip 501 displays a share summary 510. The share summary 510 includes the regular share dollar amount 511, the special share dollar amount 512, and a total share dollar amount 513. The online share slip also includes a clickable web link for the sending member to indicate their intention to send the share through paper mail or an e-service. The sending member selects either the paper check by mail select 520 or the e-service select 525. When the member clicks on paper check by mail select 520, a text field allows the sending member to transpose a check number and confirm that the share has been sent via postal mail. If the member clicks on the e-service select 525, a confirmation portion is displayed with pre-filled information. As shown in FIG. 6, the confirmation portion 619 shows the E-Service select 625 as having been selected. The selected e-service name 627 is displayed to confirm to the user that the appropriate e-service is selected. The URL for the receiving member page 628 with the selected e-service is displayed. The member is responsible to confirm payment sent, in a similar fashion to confirming sending the postal mail. The total share dollar amount is prefilled in an editable total share dollar amount field 630. The current date is prefilled in an editable current date field 631. The sending member is also presented with a subsequent web link 640. Activation of subsequent web link 640 causes instructions to be displayed for the selected e-service. If the member has selected the wrong e-service or otherwise wishes to cancel sending the share, there is a cancel button 641. Clicking the subsequent web link 640 may open up an instruction display 701, as shown in FIG. 7. The instruction display 701 presents the share sending member with instructions 702 specific to the selected e-service platform and how to return to the sharing organization member portal to confirm electronic transfer of the share. If the member understands and agrees with the instructions, there is a confirmation button 703. If the member does not agree with the instructions, the member may still cancel the transfer 704. In this embodiment, the confirmation button 703 is the clickable web link that sends the share sending member to the e-service portal to send the share to the receiving member. The URL destination associated with the confirmation button 703 may be hard coded to pre-fill in the total share dollar amount. Members then send their share payment through the linked e-service. The sharing member may also send a brief note of encouragement to the Member in need assigned to them.

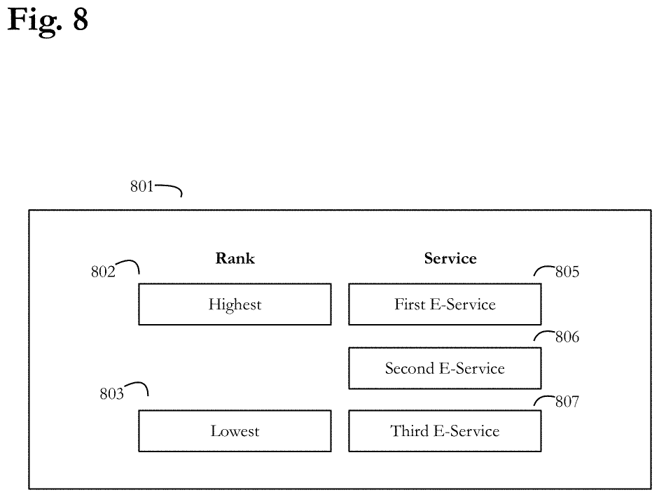

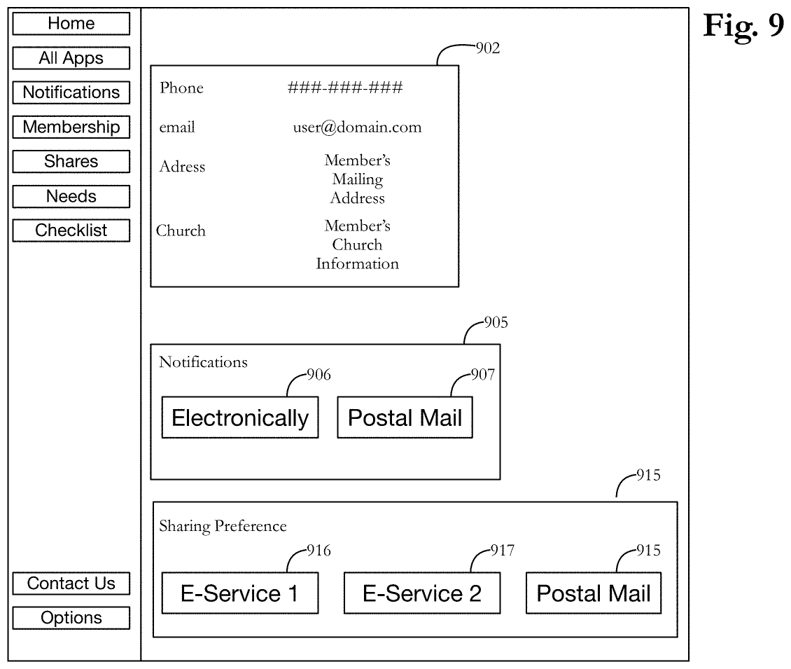

As shown in FIG. 8, the system may employ a user interface preference selection page 801 to allow the member or a member service representative to rank the service provider parameter. The member or member service representative may select a priority rank, from highest 802 to lowest 803 for individual electronic share transfer services such as first service provider 805, second service provider 806, and third service provider 807.

Alternatively, as shown in FIG. 9, the member accesses a graphical user interface 901 to select their e-service preference. Graphical user interface 901 displays member biographical data 902, such as member's name, address, email, phone number, and church affiliation. A notification section 905 allows the member to select between electronic communication preference 906 and postal mail communication preference 907. A sharing preference pane 915 allows the member to select a preferred e-service: a first e-service 916, a second e-service 917, and postal mail 918. When a member selects an e-service, the member is prompted with a subsequent screen to enter the e-service account profile information. For example, the member may enter their paypal.me link. The link information is used to generate a clickable web link when the member has a need shared to a sending member also enrolled in the e-service or who opts to receive electronic share slips.

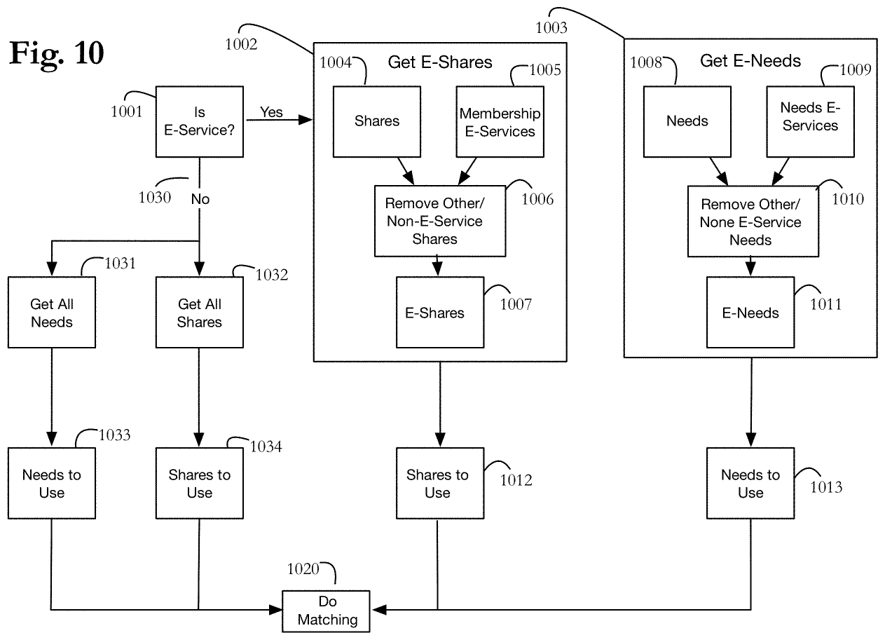

The queuing process for e-service and regular needs and shares is shown in more detail in FIG. 10. The allocation engine determines whether each need is an e-service need and whether each share is an e-service share, according to step 1001. If yes, the allocation engine proceeds to get e-shares, according to step 1002, and to get e-needs, according to step 1003. In Step 1002, shares 1004 and membership e-services 1005 are combined. From this combination, any other non-e-service shares are removed from consideration, according to step 1006. This results in only e-shares 1007. In Step 1003, needs 1008 and needs e-services 1009 are combined. From this combination, any other non-e-service needs are removed from consideration, according to step 1010. This results in only e-needs 1011. The allocation engine selects the e-shares 1007 for processing according to step 1012. The allocation engine selects the e-needs 1011 for processing according to step 1013. The allocation engine performs the matching on these selected e-shares and selected e-needs, according to step 1020. Once the allocation begins to process regular needs or shares, the allocation engine proceeds down pathway 1030. The allocation engine gets all needs 1031, which includes regular needs and any remaining e-service needs. The allocation engine also gets all shares 1032, which includes regular shares, any remaining e-service shares, and online share slip shares. The allocation engine selects all needs 1031 for processing, according to step 1033. The allocation engine selects all shares 1032 for processing, according to step 1034. The allocation engine performs the matching on these selected all shares and selected all needs, according to step 1020.

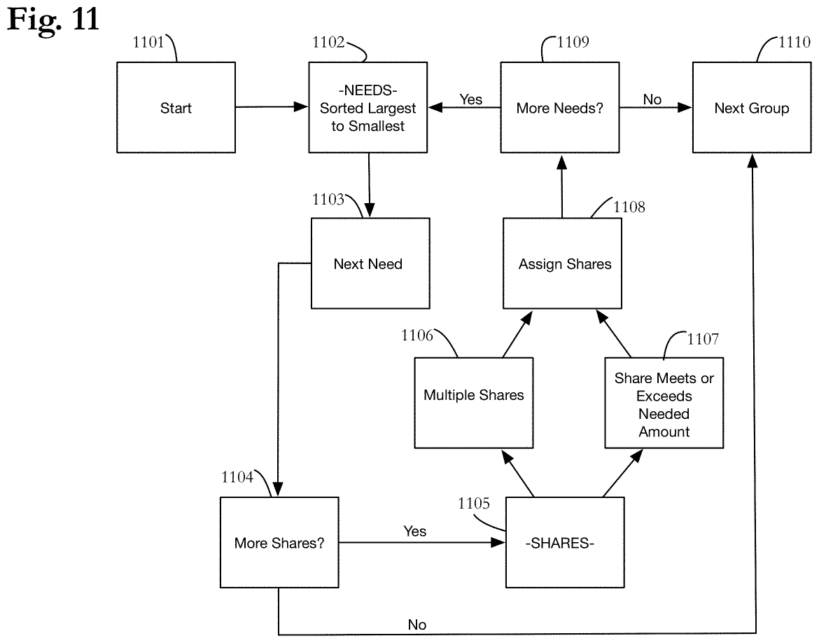

A process for matching needs is shown in FIG. 11. The same matching process may be used for both special needs and regular needs. The allocation may match the needs as separate groups, special needs with special shares, and regular needs with regular shares, by attributing an appropriate parameter for the appropriate needs and shares. Upon starting the matching process 1101, the needs are sorted by dollar amount from largest to smallest. The largest remaining need is selected 1103. If there are more shares 1104, then the shares queue 1105 is accessed. The allocation engine attempts to match a single share that meets or exceeds the need amount 1107. If the allocation is unable to match a single share that meets or exceeds the need amount 1107, then the allocation engine makes a tentative match of multiple shares 1106. The need is either met through multiple shares 1106 or a single share that meets or exceeds the need amount 1107, and then those shares are assigned to that selected need. The system continues to assess needs 1109. If there are more needs, then the 1102-1108 cycle continues. If there are no more needs, then the system proceeds to the next group, according to step 1110. The system may also move on to the next group if step 1104 gives a result that there are no more shares for the specified group.

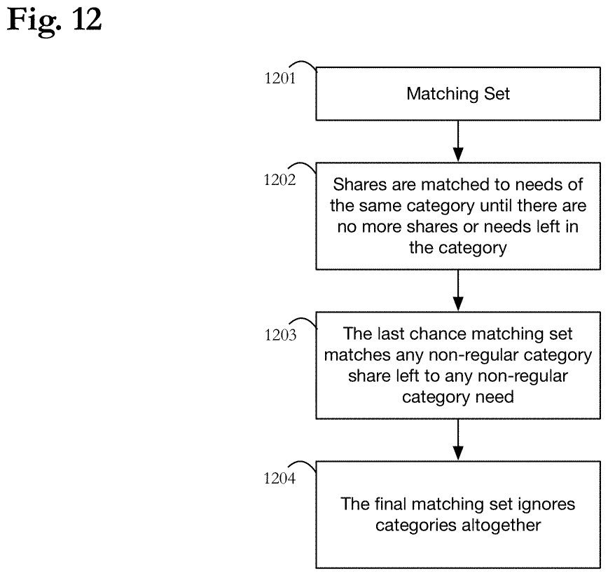

A process for a matching set 1201 is shown in more detail in FIG. 12. Shares are first matched to needs of the same category until either the shares in that category or the needs in that category are exhausted, according to step 1202. The category may be an e-service or may be another aspect of the share transaction. For example, the category may be currency, where members prefer to share in Mexican Pesos or Canadian Dollars. Needs that share the currency category may be preferentially matched with members who are setup to share in the same currency category. Additionally, electronic currency systems such as BitCoin™ and Ethereum™ combine payment networks and the form of currency. As such, electronic currency systems may be included as a category for preferential matching of needs and shares.

Once either the shares or needs of each category are exhausted, then the allocation engine may proceed to a last chance matching set, according to step 1203. During the last chance matching set, any non-regular category share is matched to any non-regular category need. In this way, more technically savvy users have a greater chance of being matched together. This last chance matching set may be more advantageous when the categories are e-services, and may be less advantageous when the categories are currencies. The final matching set ignores categories and matches any remaining need with any remaining share, according to step 1204.

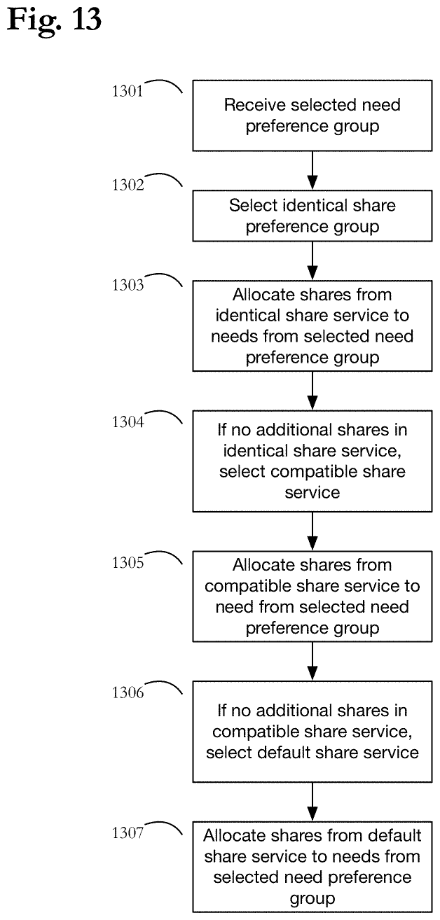

A selected need preference group may be matched with multiple compatible share preference groups, as shown in FIG. 13. The allocation engine may be configured to prioritize identical electronic share transfer services first and then proceed to secondary preferences or compatible share transfer services. A compatible share transfer service may be one that allows a generic transfer type, such as an automated clearing house request or via an application programming interface (API) that allows transfers between different e-payment systems. The allocation engine first receives a selected need preference group, as shown in step 1301. The allocation engine selects needs that share an identical share service, according to step 1302. The allocation engine then proceeds to allocate shares from identical share service to the needs from the selected need preference group, according to step 1303. If no additional shares are available in the identical share service, then the allocation engine may proceed to select a compatible share service, according to step 1304. Shares are allocated from compatible share service group to the needs from the selected need preference group, according to step 1305. If no additional shares are available in the compatible share service group, then the default share service group is selected, according to step 1306. Shares from the default share service group are allocated to the need from the default share service until the needs or shares are exhausted, according to step 1307.

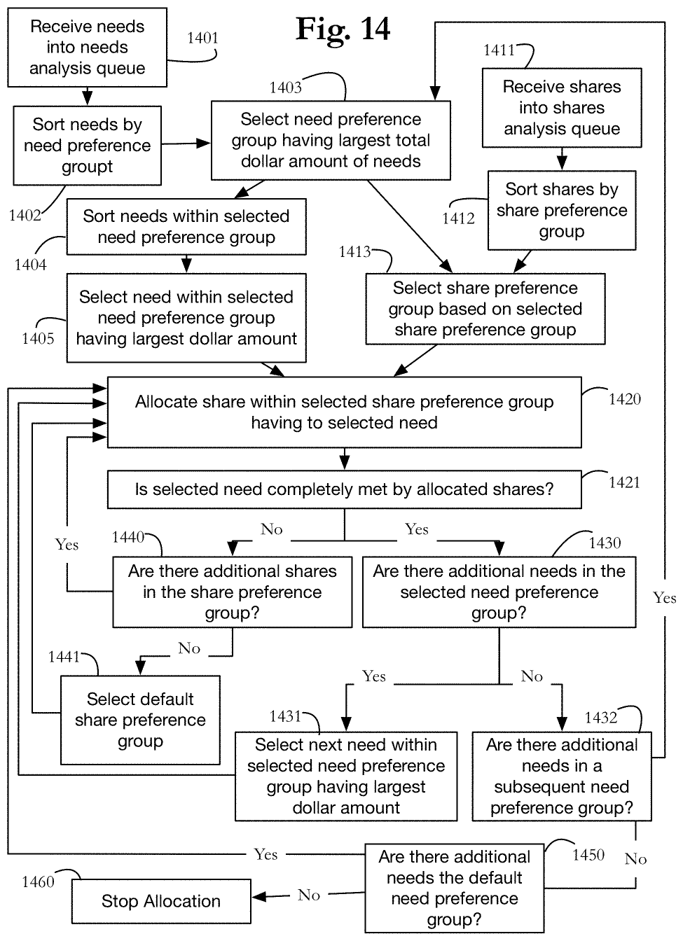

Another embodiment of the allocation engine is shown in FIG. 14. The needs qualifying for sharing under the guidelines of the sharing organization are received for a particular period into a needs analysis queue, according to step 1401. The needs analysis queue is sorted by need preference groups, according to step 1402. The allocation engine selects the needs preference group having the highest total dollar amount of needs, according to step 1403. The needs are sorted within the selected need preference group, from highest dollar amount to lowest dollar amount, according to step 1404. A need is selected within the selected need preference group that has the largest dollar amount, according to step 1405.

In order to match the needs from the selected share preference group with appropriate shares, the shares for the particular period are also received into a shares analysis queue, according to step 1411. The shares analysis queue is sorted by share preference group, according to step 1412. The allocation engine selects the share preference group based on the selected need preference group, according to step 1413. Step 1413 can incorporate the compatible categories to preferentially select the identical share preference group and then proceed to select a compatible preference group.

The selected need of step 1405 is then matched with shares from the selected share preference group of step 1413. The largest share within the selected share preference group is allocated to the selected need, unless the allocation engine identifies a share that is equal to or greater than the remaining share amount, according to step 1420. If the selected need has been completely met by the allocated shares, then the allocation engine will proceed to step 1430. If the selected need has not been completely met by the allocated shares, then the allocation engine will proceed to step 1440.

If the selected need has not been completely met by the allocated shares, then the system will determine if there are additional shares in the share preference group, according to step 1440. If there are additional shares within the selected share preference group, then the allocation engine returns to step 1420 to allocate the next largest share from the selected share preference group, unless there is a single smaller share that satisfies the remaining dollar amount of the need. If there are not any additional shares within the selected share preference group, then the system selects the default share preference group according to step 1441.

If the selected need has been completely met by the allocated shares under step 1421, then the allocation engine will check whether there are additional needs in the selected need preference group, according to step 1430. If there are additional needs, then the allocation engine will select the next need within the selected need preference group having the next largest dollar amount, according to step 1431. The system then returns to step 1420 to continue the matching of the new need with the available shares. If there are no additional needs in the selected need preference group, then the allocation checks whether there are additional needs in a subsequent need preference group having the next largest total dollar amount, according to step 1432. If there are additional needs in subsequent need preference groups, then the system returns to step 1403. If there are not additional needs in a subsequent group, then the allocation engine proceeds to check whether there are additional needs in the default need preference group, according to step 1450. If there are needs in the default need preference group, then the system returns to step 1420 to assign shares without regard to preference groupings. If there are no additional needs in the default need preference group, then the system stops the allocation process according to step 1460.

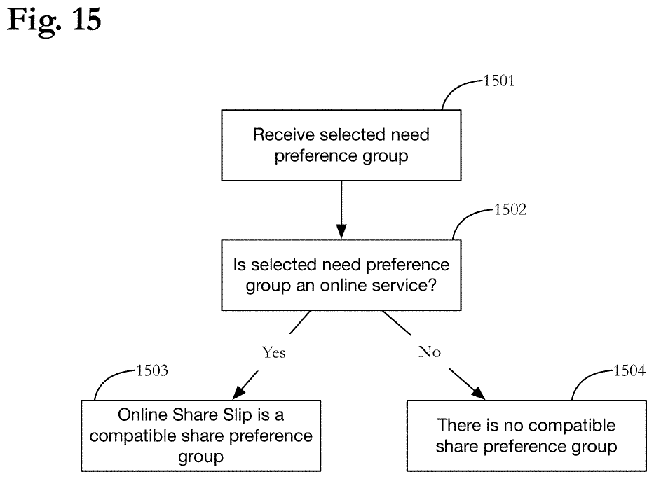

A process for determining compatible share services is shown in FIG. 15. First, the allocation engine receives a selected need preference, according to step 1501. The system determines whether the selected need preference group is an online service, according to step 1502. If the share preference group is an online service, then online share slip group is a compatible share preference group, according to step 1503. If the share preference group is not an online service, then the online share slip group is not a compatible share preference group, according to step 1504.

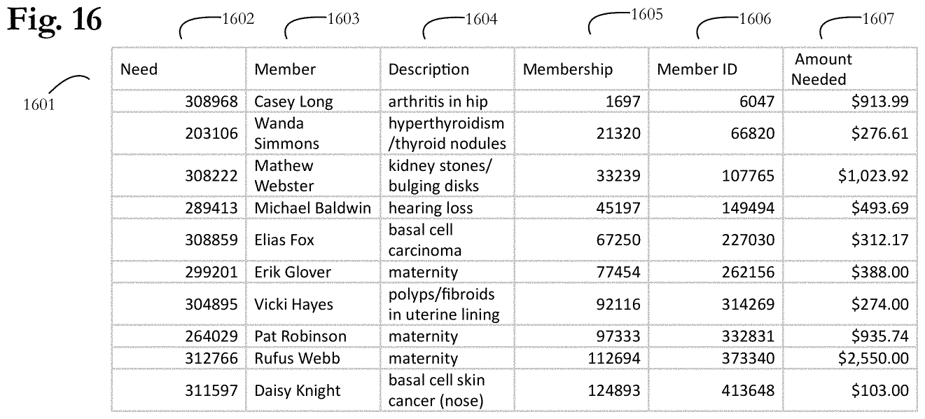

FIG. 16 shows an example data set 1601 for need submission. Needs are given a unique need identification number 1602. Each need is associated with the submitting member 1603. The data set 1601 also includes a description of the medical need 1604, membership number 1605, member ID 1606 identifying the particular member within the membership having the need, and total need amount 1607.

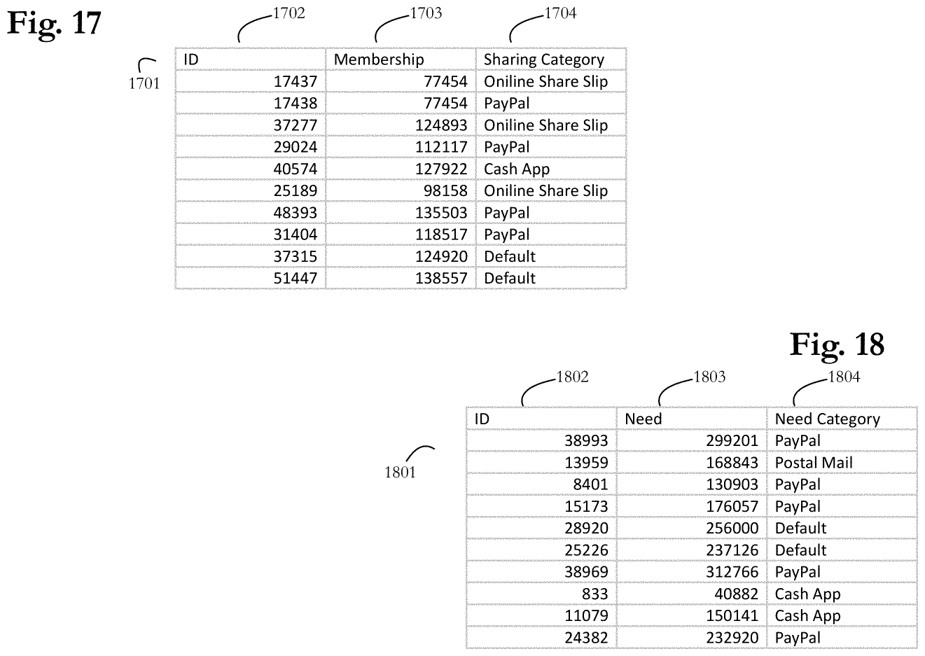

FIG. 17 shows an example sharing preference data set 1701 for storing members' sharing preferences. The data set 1701 includes a unique identification number 1702 and the membership number 1703. Each Membership is associated with one or more categories.

FIG. 18 shows an example need preference data set 1801 for storing members' need preferences. The data set 1801 includes a unique identification number 1802 and the membership number 1803. Each Membership is associated with one or more need categories.

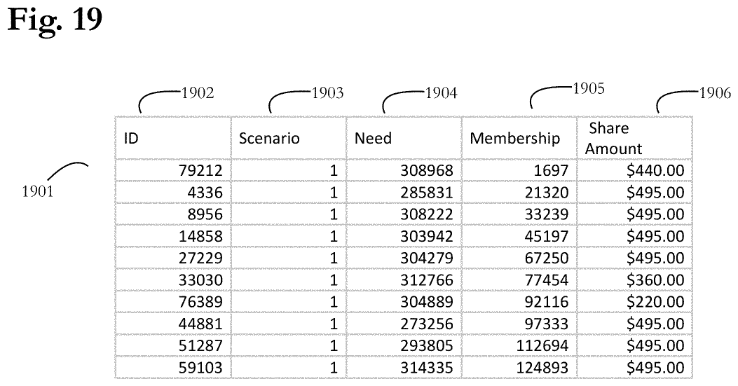

An example allocation data set 1901 from a completed run of the allocation engine is shown in FIG. 19. A unique identifier is shown in column 1902. A scenario number 1903 is recorded for each time the allocation engine is run during a period. This allows the analyst to adjust the settings and track the results the varied settings. The unique need number 1904 is included in the allocation data set 1901, along with the membership number 1905. The share amount 1906 is shown matched to the unique need number 1904. This information is used to populate the respective share slips for each member.

In order to increase the number of sharing members being connected to receiving members that have the same category, the allocation engine preferentially matches categories having the highest dollar amount. A web link is generated to direct the sharing member to an online account portal for the membership who submitted the selected need. The web link includes the share amount to reduce the opportunity for member error in transposing the share number. The online share slip is displayed to the sharing member through the online account portal.

A peer-to-peer needs sharing system allows members of the peer-to-peer needs sharing organization to periodically share in needs of other members at a fixed share amount, while optimizing the transfer time with respect to the member's preference for electronic or non-electronic share transfers. The system communicate can be configured to communicate between a shares management component, a needs management component, an allocation component, a plurality of member components, a provider component, and an e-payment service provider component.

The shares management component loads a shares analysis queue with the plurality of available shares, the share amount associated with each of the plurality of available shares, and a share preference group associated with each of the plurality of shares or with each of the members.

The needs management component may be configured to receive data regarding a plurality of need requests from multiple sources, such as a member component or a provider component. Need requests can also be directly entered into the needs management component. The needs management component loads a needs analysis queue with the plurality of need requests from the members, a need amount for each of the plurality of need requests, and a needs preference group associated with each of the plurality of need requests.

The allocation component communicates with the shares management component and the needs management component to assign the shares from the shares analysis queue to the needs in the needs analysis queue. The allocation component allocates the plurality of available shares from the shares analysis queue to the plurality of need requests by selecting a first need request from the needs analysis queue. The allocation component assigns a first share that has a share preference group that is compatible with the first need request. The allocation component may select based on the amount of a first share, such as by selecting the share amount of the first share is the greatest in the shares analysis queue without exceeding the need amount for the first need.

Each component may be configured with a graphical user interface shown on a display device to display information such as need amount, share amount, receiving member's biographical information, selected share preference group, outbound share assigned to a selected member, and other data points discussed above. The display device of the member's component displays an online share slip generated by the peer-to-peer needs sharing system. The online share slip has a clickable web link for the e-payment service. The first clickable web link may be encoded with the receiving member's identity and the share amount of the first share. The display device may also display a second clickable web link configured to report to the peer-to-peer needs sharing system that the share has been sent via postal mail, in this way a sending member who is configured to send a share through electronic services but who is matched with a receiving member having a preference for paper checks may electronically confirm that the sending member's share has been sent. The display device may also display a plurality of service providers, as indicated above, for a selecting member to select the share preference group to associate with the selecting member's share.

The peer-to-peer needs sharing system may also comprise an electronic share transfer service compatibility list indicating the compatibility of a plurality of electronic share transfer services from which of the share preference group and the needs preference group are selected.

It is understood that other embodiments will become readily apparent to those skilled in the art from the following detailed description, wherein various embodiments are shown and described by way of illustration only. As will be realized, the concepts are capable of other and different embodiments and their several details are capable of modification in various other respects, all without departing from the spirit and scope of what is claimed as the invention. Accordingly, the drawings and detailed description are to be regarded as illustrative in nature and not as restrictive.

I claim:

1. A method for generating an online share slip with a clickable web link for a plurality of members of a peer-to-peer needs sharing community, the method comprising the steps of:
a. assigning a share preference group and a share amount to each of the plurality of members of the peer-to-peer needs sharing community corresponding to the member's preferred method of sending an outbound share;
b. receiving a plurality of need amounts from a plurality of needing members of the peer-to-peer needs sharing community;
c. assigning a needs preference group to each of the plurality of needing members corresponding to each of the plurality of needing members' preferred method of receiving the share amount;
d. loading the share amount and the share preference group for each of the plurality of members of the peer-to-peer needs sharing community into a shares analysis queue;
e. loading the plurality of need amounts and the needs preference group for each of the plurality of needing members into a needs analysis queue;
f. allocating the shares analysis queue in an iterative process comprising the following steps for each of the plurality of need amounts in the needs analysis queue:
i. selecting a selected need from the needs analysis queue corresponding to a first needing member from the plurality of needing members;
ii. assigning an assigned share from the shares analysis queue corresponding to a first sending member of the plurality of members of the peer-to-peer needs sharing community, wherein the share preference group of the first sending member is compatible with the needs preference group of the first needing member; and
g. generating the online share slip for each of the plurality of members of the peer-to-peer needs sharing community having the share preference group corresponding to an e-payment service, wherein the online share slip displays the clickable web link for the e-payment service.

2. The method of claim 1 further comprising the steps of:
a. printing a paper share slips for each of the plurality of members of the peer-to-peer needs sharing community having the share preference group corresponding to a paper check; and
b. delivering the paper share slips by postal mail.

3. The method of claim 1, wherein the selected need is the largest need amount in the needs analysis queue.

4. The method of claim 3, wherein the assigned share has the share amount that is the largest in the shares analysis queue that is compatible with the needs preference group of the first needing member.

5. A peer-to-peer needs sharing system for a plurality of members to periodically share in needs of other members at a fixed share amount, the peer-to-peer needs sharing system comprising:
a. a shares management component for loading a shares analysis queue with a plurality of available shares, a share amount for each of the plurality of available shares, and a share preference group associated with each of the plurality of available shares;
b. a needs management component for receiving a plurality of need requests and for loading a needs analysis queue with the plurality of need requests, a need amount for each of the plurality of need requests, and a needs preference group associated with each of the plurality of need requests; and
c. an allocation component for allocating the plurality of available shares from the shares analysis queue to the plurality of need requests by selecting a first need request from the needs analysis queue, and assigning a first share having the share preference group compatible with the first need request, wherein the share amount of the first share is the greatest in the shares analysis queue without exceeding the need amount for the first need request.

6. The peer-to-peer needs sharing system of claim 5, further comprising:
a. a display device for displaying a share slip having a first clickable web link for an e-payment service.

7. The peer-to-peer needs sharing system of claim 6, wherein the first clickable web link is encoded with a receiving member's identity and the share amount of the first share.

8. The peer-to-peer needs sharing system of claim 7, wherein the display device is also for displaying a second clickable web link configured to report to the peer-to-peer needs sharing system that the share has been sent via postal mail.

9. The peer-to-peer needs sharing system of claim 8, wherein the display device is also for displaying a plurality of service providers for selecting the share preference group associated with a selected available share associated with a selecting member.

10. The peer-to-peer needs sharing system of claim 9, further comprising:
a. an electronic share transfer service compatibility list indicating compatibility of a plurality of electronic share transfer services from which of the share preference group and the needs preference group are selected.

11. The peer-to-peer needs sharing system of claim 10, wherein the allocation component is also for prioritizing allocation of the plurality of available shares from the shares analysis queue if the first need request has an electronic share transfer service that is identical, and then proceeding to allocate the plurality of available shares from the shares analysis queue if the electronic share transfer services are compatible with the electronic share transfer service of the first need request according to the electronic share transfer service compatibility list.

12. A method comprising:
a. loading a share amount and a share preference group for each of a plurality of members into an shares analysis queue;
b. receiving a need preference group and a need amount for a plurality of needing members;
c. loading the need preference group and the need amount into a needs queue; and
d. proceeding, starting with the shares analysis queue and the needs queue, to (1) selecting a first need from the needs queue having a first need-preference group, (2) selecting a selected share from the shares analysis queue if the share preference group of the selected share is compatible with the need preference group, and (3) assigning the selected share to an assigned shares list for a first receiving member, wherein substeps 1-3 are performed in an iterative manner until the earlier of (1) there are no more needs in the needs queue having a first need-preference group or (2) there are no more share-amounts in the shares analysis queue having the share preference group compatible with the need preference group.

13. The method of claim 12, further comprising:
a. generating an online share slip for the plurality of members having a clickable web link for an e-payment service.

14. The method of claim 12, further comprising:
a. generating an online share slip for the plurality of members having a clickable web link for an e-payment service only if the need preference group is an online service.

15. The method of claim 12, wherein the selected share is selected only if the selected share has the share amount that is the largest in the shares analysis queue that is compatible with the needs preference group of the first receiving member and does not exceed a remaining need amount, where the remaining need amount is the difference between the need amount and a sum of the assigned shares list.

16. The method of claim 12, wherein the first need preference group has a greater total dollar than a second need preference group.

17. The method of claim 12, further comprising:
a. proceeding to a subsequent iterative process step for a second need-preference group.

18. The method of claim 17, further comprising:
a. matching the shares analysis queue to the needs queue without regarding the need preference group as a final matching step.
